(19) United States
(12) Patent Application Publication
(10) Pub. No.: US 2014/0357620 A1
(43) Pub. Date: Dec. 4, 2014

(54) METHOD FOR ADMINISTRATION OF A GAMMA SECRETASE INHIBITOR

(71) Applicant: Hoffmann-La Roche Inc., Nutley, NJ (US)

(72) Inventors: John Frederick Boylan, Bedminster, NJ (US); Stanislaw M. Mikulski, Essex Fells, NJ (US)

(21) Appl. No.: 14/464,056

(22) Filed: Aug. 20, 2014

Related U.S. Application Data
(63) Continuation of application No. 13/400,280, filed on Feb. 20, 2012, now abandoned.
(60) Provisional application No. 61/448,216, filed on Mar. 2, 2011.

(51) Int. Cl. A61K 31/55

(52) U.S. Cl. CPC A61K 31/55
USPC 514/212.04

(57) ABSTRACT

There are provided a new dosage regimens for the gamma secretase inhibitor 2,2-Dimethyl-N-((S)-6-oxo-6,7-dihydro-5H-dibenzo[b,d]azepin-7-yl)-N-(2,2,3,3,3-pentafluoro-propyl)-malonamide which maximizes anti-tumor activity while maintaining acceptable toxicity levels.

### PRIORITY TO RELATED APPLICATION(S)

This application claims the benefit of U.S. Provisional Application No. 61/448,216, filed Mar. 2, 2011, which is hereby incorporated by reference in its entirety.

### FIELD OF THE INVENTION

The present invention is directed to improved methods of administration of Gamma Sectretase Inhibitor (Compound A) in the treatment of cancer. In particular, the invention is directed to improved methods of administration of said Inhibitor that provide desirable antineoplastic effects with a tolerable level toxicity. The methods of the invention are characterized by administering Compound A in a variety of schedules over different days per cycle.

### BACKGROUND OF THE INVENTION

Cancer is a disease characterized by uncontrolled proliferation. Advances in understanding the signals that drive cancer are being made. During development and tissue remodeling, pluripotent stem cells serve as the source for differentiating cells to give rise to non-proliferating specialized cell types. A link between the characteristics of these stem cells and the rapid uncontrolled proliferation of tumors is becoming clear. One of the major developmental signaling axes is the Notch pathway. Notch signaling regulates cell-fate by mediating the differentiation of progenitor cells during development and self-renewal of adult pluripotent stem cells. Notch functions to maintain progenitor cells in a pluripotent rapidly proliferating state. The Notch pathway plays an important role in development differentiation and processes of hematopoiesis and lymphopoiesis. It is involved in generation, proliferation and differentiation of hematopoietic stem cells during embryonic development.

Notch gene amplification, chromosomal translocation or mutations lead to elevated Notch signaling, thereby imparting a tumor growth advantage by keeping tumor cells in a stem cell-like proliferative state. Therefore, there is a very strong correlation between mutation in the Notch signaling pathway and pathogenesis of malignancies.

The Notch proteins, represented by four homologs in mammals (Notch1, Notch2, Notch3, and Notch4), interact with ligands Delta-like 1, Delta-like 3, Delta-like 4, Jagged 1, and Jagged 2. After ligand binding, Notch receptors are activated by serial proteolytic cleavage events including intramembranous cleavage regulated by γ-secretase. Such a γ-secretase-processed Notch becomes active as a form called ‘intracellular Notch’ (ICN). The ICN translocates to the nucleus and forms part of a large transcription complex involving the CSL (CBF-1, Suppressor of hairless, Lag) transcriptional regulator directly altering the expression of key proliferation- and differentiation-specific genes.

The Gamma Secretase Inhibitor 2,2-Dimethyl-N-((S)-6-oxo-6,7-dihydro-5H-dibenzo[b,d]azepin-7-yl)-N′-(2,2,3,3,3-pentafluoro-propyl)-malonamide (disclosed in WO 2005/023772 as useful for the treatment of Alzheimer's disease) is a water-soluble, orally-administered small molecule antagonist of γ-secretase, a key enzyme in the intramembrane proteolytic processing of several signaling receptors, including Notch, amyloid precursor protein (APP), CD44, and Her4. Blocking Notch signaling via γ-secretase inhibition produces a slower growing, less transformed phenotype in human cancer cells in vivo.

As used herein, the term “Compound A ” shall refer to 2,2-dimethyl-N-((S)-6-oxo-6,7-dihydro-5H-dibenzo[b,d]azepin-7-yl)-N′-(2,2,3,3,3-pentafluoro-propyl)-malonamide or a pharmaceutically acceptable salt thereof.

The compound has the structure shown below in formula I,

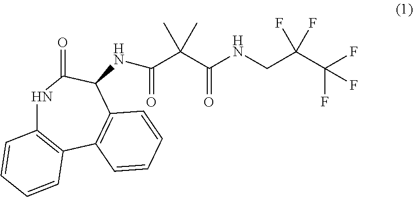 [SMILES: CC(C)(C(=O)NCC(F)(F)C(F)(F)F)C(=O)N[C@@H]1C(=O)Nc2ccccc2-c2ccccc21]

### SUMMARY OF THE INVENTION

The present invention relates to a method of treating a patient suffering with cancer, in particular a solid tumor cancer, comprising administering to the patient a compound of the formula

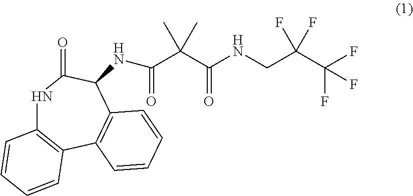 [SMILES: CC(C)(C(=O)NCC(F)(F)C(F)(F)F)C(=O)N[C@@H]1C(=O)Nc2ccccc2-c2ccccc21]

or a pharmaceutically acceptable salt thereof
following a drug regimen schedule selected from the group consisting of
(a) from about 120 mg to about 270 mg of the compound of formula 1 or a pharmaceutically acceptable salt thereof for 3 days-on; 4 days-off; for 2 weeks, q3w, 6 dosing days in a 3 week cycle;
(b) from about 80 mg to about 130 mg of the compound of formula 1 or a pharmaceutically acceptable salt thereof for 7 days-on; 14 days-off; q3w, 7 dosing days in a 3 week cycle;
(c) from about 10 mg to about 270 mg of the compound of formula 1 or a pharmaceutically acceptable salt thereof for 1 day-on; I day-off, q3w, 11 dosing days in a 3-week cycle;
(d) from about 30 mg to about 1300 mg of the compound of formula 1 or a pharmaceutically acceptable salt thereof for 1 day-on; 6 days-off; q3w, 3 dosing days in a 3-week cycle;
(e) from about 10 mg to about 600 mg of the compound of formula 1 or a pharmaceutically acceptable salt thereof for 1 day-on; 2 days-off; then 1 day-on; 3 days-off; q3w, 6 dosing days in a 3 week cycle;
and from about 5 mg to about 400 mg of the compound of formula 1 or a pharmaceutically acceptable salt thereof for 5 days-on; 2 days-off; q3w, 15 dosing days in a 3-week cycle.

### DETAILED DESCRIPTION OF THE INVENTION

As used herein the term “anti-neoplastic” means inhibiting or preventing the development, maturation or proliferation of malignant cells.

The term “therapeutically effective” means an amount of drug, or combination or composition, which is effective for producing a desired therapeutic effect upon administration to a patient, for example, to stem the growth, or result in the shrinkage, of a cancerous tumor. “q3w” means every 3 weeks.

“Therapeutic index” is a well-recognized term of art and is an important parameter in the selection of anticancer agents for clinical trial. Therapeutic Index takes into consideration the efficacy, pharmacokinetics, metabolism and bioavailability of anticancer agents. See, e.g., J. Natl. Cancer Inst. 81(13): 988-94 (Jul. 5, 1989).

The term “pharmaceutically acceptable,” such as pharmaceutically acceptable carrier, excipient, etc., means pharmacologically acceptable and substantially non-toxic to the subject to which the particular compound is administered. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium, and quaternary ammonium hydroxides, such as for example, tetramethylammonium hydroxide. The term “pharmaceutically acceptable ester” of a compound means a conventionally esterified compound having a carboxyl group, which esters retain the biological effectiveness and properties of the compound. Chemical modification of a pharmaceutical compound (i.e., drug) into a salt is a technique well known to pharmaceutical chemists to obtain improved physical and chemical stability, hydroscopicity, and solubility of compounds. See, e.g., H. Ansel et. al., Pharmaceutical Dosage Forms and Drug Delivery Systems (6^{th} Ed. 1995) at pp. 196 and 1456-1457.

The term “tumor control” means that the size of the tumor has either decreased, or has not increased by the defined and generally accepted criteria: e.g., the sum of the longest dimensions of the measurable tumor lesions has not increased by 20% or more as compared with the baseline, or with the shortest dimension of that lesion achieved post-treatment (RECIST=Response Evaluation Criteria in Solid Tumors, rules 1.1 published January 2009), or for specific tumor lesions such as those related to lymphoma and/or intracranial metastases of solid tumors, the sum of the products of the perpendicular diameters of measurable lesions has not increased by 25% or more from the baseline or from the last measurement. (See, e.g., World Health Organization (“WHO”) Handbook for Reporting Results of Cancer Treatment, Geneva (1979).

In certain instances, the criteria for the volumetric (three-dimensional =3D) tumor measurements might be applied (e.g., for the brain metastatic lesions).

“Tumor volume (in cubic millimeter)” for purposes of measuring tumor size is calculated using the ellipsoid formula:

(D×(d²))/2

where “D” represents the large diameter of the tumor, and “d” represents the small diameter.

The term “autoinduction” shall mean a promotion of the compound's own metabolism by Compound A inducing the activity of the relevant CYP450 metabolizing enzyme(s).

In the first two embodiments of the invention, the 3-4 schedule (3 days-on; 4 days-off; for 2 weeks, q3w) (A) and the 7-14 schedule (7 days-on; 14 days-off; q3w) (B) both contain rest weeks in the 3-week cycle. This will enable the collection of PD data using these two schedules. These two schedules (A and B) include 6 and 7 dosing days in a 3-week cycle, respectively.

In yet another embodiment of the invention, the 1-1 schedule (1 day-on; I day-off q3w) (schedule C) provides a simple schedule and is relevant considering that the mean terminal half-life defined thus far in an ongoing first phase 1 trial of Compound A is reported to be 42.2 hours (range between 10 to 93 hours). This schedule (C) includes 11 dosing days in a 3-week cycle.

In another embodiment of the invention the 1-6 schedule (1 day-on; 6 days-off; q3w) (schedule D) will assess the relevance of a weekly administration of Compound A. Because it does not include any consecutive dosings, this simple regimen should minimize the risk of auto-induction. Moreover, it should allow assessment of maximal concentration (Cmax) effect on toxicity and efficacy. It incorporates 3 dosing days in a 3-week cycle.

In another embodiment of the invention the 1-2-1-3 schedule (1 day-on; 2 days-off; then 1 day-on; 3 days-off; q3w) (schedule E) represents an intermediate intermittent regimen between the 1-6 schedule and the 1-1 schedule. It integrates Compound A administration twice a week to limit the risk of autoinduction while increasing systemic exposure with respect to the 1-6 schedule. It includes 6 dosing days in a 3-week cycle.

In another embodiment of the invention the 5-2 schedule (5 days-on; 2 days-off; q3w) (schedule F) represents the most dose intense schedule to be tested in this study. In this schedule (F), patients are to be dosed for 5 consecutive days with 2 days off each week, without any rest weeks. It comes close to a continuous administration schedule which was investigated with success in a preclinical model (Teachey, Seif et al. 2008). Moreover, it will allow assessment of safety of a regimen that could be used concommitently with radiation treatment. It includes 15 dosing days in a 3-week cycle.

Although the different schedule schedules (A to F) include an increasing numbers of dosing days (from 3 to 15 dosing days in a 3-week cycle), the initial drug doses will be adapted so that the dose intensity of Compound A given in a 3-week cycle does not exceed the one offered by the 3-4 schedule at the highest safe dose tested so far (270 mg/day*6 dosing days every 3 weeks in 3-4 schedule). If deemed safe, then dose escalation to dose intensities at and above that given in the 3-4 schedule at 270 mg/day will be performed. For safety reasons, the first cohorts of patients enrolled in schedules C, D, E and F will be treated with lower doses (dose level 1: 14% to 37% of dose intensity of 3-4 schedule at 270 mg). Then in the subsequent cohorts, the dose will be escalated by 50%-100%.

The objective of experiment 1 in the table of Example 1 was to compare six new dosing schedules of Compound A with different clinical schedules at different dose levels and determine treatment safety, efficacy, PK and PD parameters.

### EXAMPLE 1

TABLE 1
Schedules of administration and drug dose levels

| | | Total | | | | | | | | | | | | | |
|---|---|---|---|---|---|---|---|---|---|---|---|---|---|---|---|
| | | treatmen | | | | | | | | | | | | No. of | |
| | Name of | days per | Schedule of | Compound dose levels (mg) | | | | | | | | | | pts per | Cycle |
| Schedule | schedule | cycle | administration | −2 | −1 | 1 | 2 | 3 | 4 | 5 | 6 | 7 | 8 | schedule | length |
| A | 3-4 | 6 days | 3 days out of 7 | 120 | 18 | 270 | | | | | | | | 6 | 3 wks |
| | schedule | | (3 days-on; 4 | | | | | | | | | | | | (21 |
| | | | days-off) | | | | | | | | | | | | days) |
| | | | for the first 2 | | | | | | | | | | | | |
| | | | weeks | | | | | | | | | | | | |
| B | 7-14 | 7 days. | 7 days out of | 80 | 12 | 130 | | | | | | | | 6 | |
| | schedule | | 14 | | | | | | | | | | | | |
| | | | (7 days-on; 14 | | | | | | | | | | | | |
| | | | days) | | | | | | | | | | | | |
| | | | days-off) | | | | | | | | | | | | |
| C | 1-1 | Average | 1 day out of 2 | — | 10 | 20 | 30 | 50 | 80 | 120 | 180 | 270 | +50% | 3* | |
| | schedule | 11 days | (1 day-on; 1 | | | | | | | | | | | | |
| | | | day-off) | | | | | | | | | | | | |
| D | 1-6 | 3 days | 1 day per week | 30 | 60 | 120 | 180 | 270 | 400 | 600 | 900 | 1300 | +50% | 3* | |
| | schedule | | (1 day-on; 6 | | | | | | | | | | | | |
| | | | days-off) | | | | | | | | | | | | |
| E | 1-2-1-3 | 6 days | 2 days per | 10 | 20 | 40 | 80 | 120 | 180 | 270 | 400 | 600 | +50%% | 3* | |
| | schedule | | week | | | | | | | | | | | | |
| | | | (1 day-on; 2 | | | | | | | | | | | | |
| | | | days-off; 1 day- | | | | | | | | | | | | |
| | | | on; 3 days-on | | | | | | | | | | | | |
| F | 5-2 | 15 days | 5 days per | 5 | 10 | 20 | 40 | 80 | 120 | 180 | 270 | 400 | +50% | 3* | |
| | schedule | | week | | | | | | | | | | | | |
| | | | (5 days-on; 2 | | | | | | | | | | | | |
| | | | days-off) | | | | | | | | | | | | |

*More patients may be added as necessary.

In the above clinical trial, the safety, efficacy, PK and PD parameters of six intermittent administration schedules of Compound A are investigated.

Patients enrolled in the above studies have a variety of tumors including solid tumors such as, non-small cell lung cancer, various subtypes of breast cancer, colorectal cancer, prostate cancer, pancreatic cancer, melanoma, various sarcomas and primary brain tumors. Also patients with blood cancer such as leukemia are included

1. A method of treating a patient having cancer which comprises administering to said patient a compound of the formula 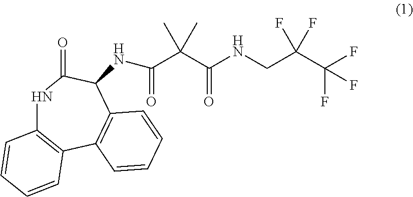 [SMILES: CC(C)(C(=O)NCC(F)(F)C(F)(F)F)C(=O)N[C@@H]1C(=O)Nc2ccccc2-c2ccccc21]
or a pharmaceutically acceptable salt thereof
following a regimen selected from the group consisting of
(a) from about 10 mg to about 270 mg per dosing day of the compound of formula 1 or a pharmaceutically acceptable salt thereof for 1 day-on; I day-off, q3w, 11 dosing days in a 3-week cycle;
(b) from about 30 mg to about 1300 mg per dosing day of the compound of formula 1 or a pharmaceutically acceptable salt thereof for 1 day-on; 6 days-off; q3w, 3 dosing days in a 3-week cycle;
(c) from about 10 mg to about 600 mg per dosing day of the compound of formula 1 or a pharmaceutically acceptable salt thereof for 1 day-on; 2 days-off; then 1 day-on; 3 days-off; q3w, 6 dosing days in a 3 week cycle; and
(d) from about 5 mg to about 400 mg per dosing day of the compound of formula 1 or a pharmaceutically acceptable salt thereof for 5 days-on; 2 days-off; q3w, 15 dosing days in a 3-week cycle.

2. (canceled)

3. (canceled)

4. The method of claim 1 wherein the patient is administered) from about 10 mg to about 270 mg per dosing day of the compound of formula 1 or a pharmaceutically acceptable salt thereof for 1 day-on; I day-off, q3w, 11 dosing days in a 3-week cycle.

5. The method of claim 1 wherein the patient is administered from about 30 mg to about 1300 mg per dosing day of the compound of formula 1 or a pharmaceutically acceptable salt thereof for 1 day-on; 6 days-oft q3w, 3 dosing days in a 3-week cycle.

6. The method of claim 1 wherein the patient is administered from about 10 mg to about 600 mg per dosing day of the compound of formula 1 or a pharmaceutically acceptable salt thereof for 1 day-on; 2 days-off; then 1 day-on; 3 days-oft q3w, 6 dosing days in a 3 week cycle.

7. The method of claim 1 wherein the patient is administered from about 5 mg to about 400 mg per dosing day of the compound of formula I or a pharmaceutically acceptable salt thereof for 5 days-on; 2 days-off; q3w, 15 dosing days in a 3-week cycle.
